(19) United States
(12) Patent Application Publication
(10) Pub. No.: US 2014/0134600 A1
(43) Pub. Date: May 15, 2014

(54) BIOMARKERS ASSOCIATED WITH DEVELOPMENT OF HEPATOCELLULAR CARCINOMA IN PATIENTS WITH HEPATITIS B VIRUS INFECTION, AND METHOD FOR DETECTION THEREOF

(71) Applicant: Amoy Diagnostics Co., Ltd., Xiamen (CN)

(72) Inventors: Li RUAN, Xiamen (CN); Hailong ZHANG, Xiamen (CN); Jibin WANG, Xiamen (CN); Limou ZHENG, Xiamen (CN)

(73) Assignee: Amoy Diagnostics Co., Ltd., Xiamen (CN)

(21) Appl. No.: 13/670,186

(22) Filed: Nov. 6, 2012

(51) Int. Cl. C12Q 1/70

(52) U.S. Cl. CPC C12Q 1/706
USPC 435/5

(57) ABSTRACT

The present invention is intended to provide a method for predicting risk of hepatocellular carcinoma (HCC) in hepatitis B virus (HBV)-infected patients with a high accuracy. More specifically, the invention provides a method for detecting eight mutations of HBV genome associated with predisposition to HCC, comprising: C1653T, A1762T, G1764A, T1674C, T1753C, C3116T, T53C and A1846T mutations, and primers and probes sets used thereof consist of SEQ ID NO: 1-SEQ ID NO: 24.

### BACKGROUND OF THE INVENTION

Infection with Hepatitis B virus (HBV) is a major public health problem, with more than 350 million HBV carriers estimated worldwide, and approximately 45% of the world's population lives in regions where HBV infection is endemic (Ganem D et al., N Eng J Med 2004; 350:1118-1129). Chronic HBV infection is one of the most important determinants of the occurrence of hepatocellular carcinoma (HCC) and liver cirrhosis (LC). Principal risk factors of development of HCC are chronic infection with HBV, HCV and liver cirrhosis. It has been established that 80% of HCC worldwide is etiologically associated with HBV (Yu M C et al., Can J Gastroenterol. 2000; 14 (8): 703-709).

HBV, a member of the family Hepadnaviridae, is a partially double-stranded DNA virus containing four overlapping open reading frames (ORFs) that encode the surface protein (S), pre-core/core, polymerase and a multifunction nonstructural protein called X.

The PreS region that consists of the PreS1 (nucleotides 2848-3204) and the PreS2 (nucleotides 3205-154) domains overlaps a region encoding the polymerase gene. The enhancer II (nucleotides 1636-1744) and basal core promoter (nucleotides 1751-1769) regions overlap with the X gene (nucleotides 1374-1835) (Kay et al., 2007; 127: 164-176).

The virus shows remarkable genetic variability and is currently classified into eight genotypes, designated A to H based on a sequence divergence greater than 8% in the entire HBV genome. Genotypes are further categorized into sub-genotypes based on nucleotide sequence divergence between 4% and 8%. HBV genotypes have distinct geographical distributions worldwide, and have been shown to differ with regard to clinical disease, prognosis and response to interferon treatment. Genotypes B and C are endemic in Asia, genotype D the Mediterranean area and Middle East, genotype E in middle Africa, genotype F in South American (Schaefer et al., J Viral Hepat. 2005; 12: 111-124; Chan et al. J Infect Dis 2005; 191:2022-2032.).

Several studies have revealed that some nucleotide mutations in the HBV PreS and Precore regions were associated with the increase risk of HCC. Some representative references were present as follow.

Laskus et al., (Laskus et al. Biochem Biophys Res Commun. 1998; 244: 812-814) reported earlier single nucleotide mutations at nucleotide A1762T/G1764A, G1896, G1899A. They studied 53 patients with HCC and 33 HBsAg positive controls. A functional part of HBV core promoter and whole precore region were sequenced directly and after cloning. HBV DNA was amplified from sera from 27 HCC patients and in all controls. As a result, fourteen (52%) patients and 12 (36%) controls were found to harbor an HBV strain with G to A transition mutation at position 1896 leading to HBeAg negative phenotype. Nine (33%) HCC patients and 2 (6%) controls harbored a mixture of wild type and HBV strains with deletions/insertions.

Baptista et al. (Baptista et al., Hepatology. 1999; 29: 946-953.) noticed successively missense mutations for nucleotides G1809T, A1762T, T1764V and C1812T in the basic core promoter in his studies from southern African patients. They found that Nucleotide divergences were higher in the basic core promoter of hepatocellular carcinoma patients when compared with asymptomatic carriers (P<0.0001). The prevalence of the nucleotides A1762T and T1764V missense mutations was 66% in patients with hepatocellular carcinoma compared with 11% in asymptomatic carriers (P<0.0001).

Ito et al. (Ito et al., Clin Infect Dis. 2006; 42: 1-7.) investigated a wide scope studies that across race and region in obtaining sample. In the study, the sera of 211 patients from different regional areas worldwide were obtained. As a result, the prevalence of T1653 was significantly higher among patients with hepatocellular carcinoma than among carriers of inactive virus who did not have hepatocellular carcinoma. Mutations in the basic core promoter region (T1762/A1764) were frequently found in all groups.

Yuan et al., (Yuan J et al., J Clin Virol. 2007; 39: 87-93) have realized much more nucleotide mutations associated with HCC development, which are nucleotide C1653T, T1762V, A1762T/G1764A, G1862T, G1888H, G1809A, and G1896. These mutations associated with HCC were V1753 and T1762/A1764; the prevalence of the V1753 was higher in HBV/C1 strains; A1898 was only found among HBV/C1, T1762/A1764 was frequently demonstrated in HBV. The T1858 (90%) and A1896 (40%) mutations were most frequent in HBV/C2.

Sung et al., (Sung et al. J Virol. 2008; 7: 3604-3611) have observed genotype-specific difference in the frequency mutations associated with risk of HCC. Studies showed that in genotype B HBV, mutations C1165T, A1762T and G1764A, T2712C/A/G, and A/T2525C were associated with HCC. HCC-related mutations T31C, T53C, and A1499G were associated with HBV subgenotype Ce, and mutations G1613A, G1899A, T2170C/G, and T2441Cwere associated with HBV subgenotype Cs.

Liu et al., (Liu et al. J Natl Cancer Inst 2009; 1066-1082.) have studied for the frequency of nucleotide mutations by statistic. They found that C1653T, T1753V, and A1762T/G1764A are associated with an increased risk of HCC. The frequencies of these mutations increase as chronic HBV infection progresses from the asymptomatic HBsAg carrier state to liver cirrhosis and HCC. Frequent examination of patients with chronic HBV infections for the presence of these mutations may be useful for identifying which patients require preventive antiviral treatment and for the prediction of HCC.

However, most of these studies in investigated association between HBV mutations and the risk of HCC had a small number of patients and often examined only specific viral mutations. Thus, the genetic point mutations in HBV increase the risk for HCC, which contributed to certain SNPs to HCC remain to be identified definitively.

### SUMMARY OF THE INVENTION

The present invention provides an effective means and method of predicting risk for HCC with high accuracy and high predictability so as to contribute the prevention, diagnosis and treatment of HCC.

In order to achieve the abovementioned objects, the inventors searched relevant literature from the database of the published medical articles. The 75 articles related to HBV nucleotide mutation associated with HCC were reviewed after identification and selection from the database. At last, 39 related articles were selected for results analysis. The most commonly reported HBV single nucleotide mutations associated with HCC risk were located in PreS region and basal core promoter region.

The present inventors have extracted 14 mutation sites that are associated with HCC based on our literature review. Among them, eight mutation sites were evaluated further by our experiments, which are C1653T, A1762T, G1764A, T1674C, T1753C, C3116T, T53C, and A1846T.

The literature review revealed 32 mutation sites associated with HCC, which are T31C, T53C, T1479C, C1485T, C1499H, G1613A, C1165T, C1653T, C1726A, T1727A, T1730C, A1752G, T1753V, A1762T, G1764A, T1765V, C1766T, C1773T, G1799C, G1809T, C1812T, T1856C, C1858T, G1862T, G1888H, G1896A, G1898A, G1899A, T2170C/G, T2441C, A/T2525C and T2712V. Seven of the mutation sites showed higher mutation frequency compared to the other 25 mutation sites. These are C1653T, T1753V, A1762T, G1764A, C1858T, G1896A, and G1899A.

The further experimental findings verify the mutations of C1653T, T1753C, A1762T and G1764A were with the highest frequency mutations, which were essential for predictive risk of HCC.

The present invention was made based on the abovementioned finding and provides the following configuration.
1. The statistical results indicated that it is useful for prediction risk of HCC by determining nucleotides A1858T, G1896A, G1899A, C1485T, C1499A, G1613A, T1753C, T1674C, C1653T, A1762T, G1764A, C3116T, T53C, and A1846T.
2. The further experiments relevant to risk of HCC provide consensus mutation sites with literatures review, which are C1653T, T1753C, A1762T and G1764A.
3. The invention provides an effective means for prediction risk of development of HCC by determining mutation sites comprise: A1858T, G1896A, G1899A, C1485T, C1499A, G1613A, T1753C, T1674C, C1653T, A1762T, G1764A, C3116T, T53C, and A1846T.
4. The present invention discovered 14 mutations (A1858T, G1896A, G1899A, C1485T, C1499A, G1613A, T1753C, T1674C, C1653T, A1762T, G1764A, C3116T, T53C, and A1846T) are associated with susceptibility to development of HCC, based on statistical analysis and further experiment estimate. Specifically, C1653T, A1762T, G1764A, T1674C, T1753C, C3116T, T53C, and A1846T have been identified as major candidate determinants of the susceptibility to HCC in human.

The present invention also provides a method for detecting eight mutations of HBV associated with susceptibility to cause of HCC, including C1653T, A1762T, G1764A, T1753C, T1674C, C3116T, T53C and A1846T, the composition comprising as follows:

| | | |
|---|---|---|
| DNA | 5 | μL |
| Primers | 1-5 | pmol |
| probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |

The present invention also provides the primers and probes for detecting eight mutations of HBV, comprising mutations C1653T, A1762T, G1764A, T1753C, T1674C, C3116T, T53C and A1846T, the primers and probes sequence consist of SEQ ID NO: 1-SEQ ID NO: 24.

The present invention also provides kits for detecting HBV eight mutations to cause HCC. In one embodiment, the kits comprise primers and probes consist of SEQ ID NO: 1-SEQ ID NO: 24 and following composition.

| | | |
|---|---|---|
| DNA | 5 | μL |
| Primers | 1-5 | pmol |
| probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |

The invention provides specific primers and probes neclueotide sequence and substantically identical nucleotide sequence thereto, which allow accurate detection of eight mutations, comprising C1653T, A1762T, G1764A, T1753C, T1674C, C3116T, T53C and A1846T.

“Identical nucleotide sequence” in the primer and probe sequence context as used herein, means hybridizaion to a target under stringent condidation, and also that the nucleotide sequence segments, when compared, are the same when properly aligned, with the appropriate nucleotide insertion and deletions, in at least 70% of the nucleotides, typically, usually, at least 90% of nucleotides.

### BRIEF DESCRIPTION OF THE DRAWINGS

FIG. 1 is a flow chart showing a strategy for selected nucleotide mutations sites: HBV=hepatitis B virus; HCC=hepatocellular carcinoma;

FIG. 2 is a graph showing statistical mutation frequency of all mutation sites in HBV all genotypes.

FIG. 3 is a graph showing statistical mutation frequency of all mutation sites in HBV B/C genotypes.

FIG. 4A is a graph illustrating mutation of PCR detection for T1753C from HCC sample.

FIG. 4B is a graph illustrating wildtype of PCR detection for T1753C from control sample.

FIG. 5A is a graph illustrating mutation of PCR detection for A1846T from HCC sample.

FIG. 5B is a graph illustrating wildtype of PCR detection for A1846T from control sample.

### DETAILED DESCRIPTION OF THE INVENTION

The present invention provides an effective means to predict which HBV-infected individuals are more likely to develop HCC, as well as a method for early diagnosis of HCC, so as to allow early treatment before HCC becomes advanced.

The inventors have identified Single Nucleotide Polymorphisms (SNPs) in the genome of HBV that are associated with risk for HCC. A SNP is the smallest unit of genetic variation, which represents a position in a genome where individuals of the same species may have different nucleotides inserted into their DNA sequences.

SNPs in the genome of HBV have been noticed previously in the peer reviewed scientific literatures. However, there is no consensus about the link between HBV SNPs and the development of HCC. The present inventors searched databases using terms “hepatitis B Virus,” “Nucleotide mutation” and “Hepatocellular Carcinoma” to find related studies.

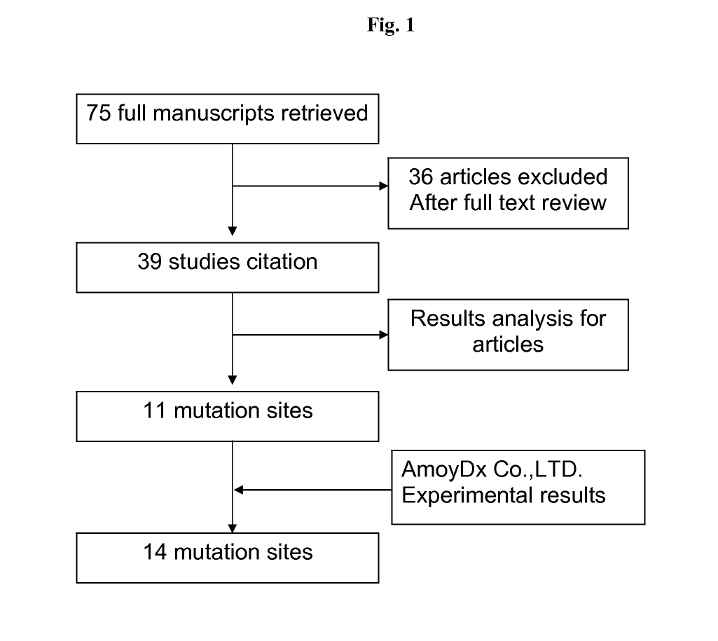

The present inventors searched and identified 1255 potentially relevant articles from databases of the published literatures, then 1001 literatures were excluded after abstract review, among 253 abstract of the literatures related to HBV nucleotide mutations associated with HCC and liver cirrhosis were reviewed, and 75 full manuscripts review relevant to HBV single nucleotide polymorphism. At last, 39 articles were selected for analysis. These 39 studies included 14 from mainland China, seven from Japan, five from Taiwan, four from the United States, two each from Korea, Vietnam and South Africa, and five from two or more countries or regions. The most commonly reported HBV single nucleotide mutations associated with HCC risk were located in PreS region and basal core promoter region. In the selecting process, the studies that were not published as full reports were excluded, The work flow chart of work idea was showed in FIG. 1.

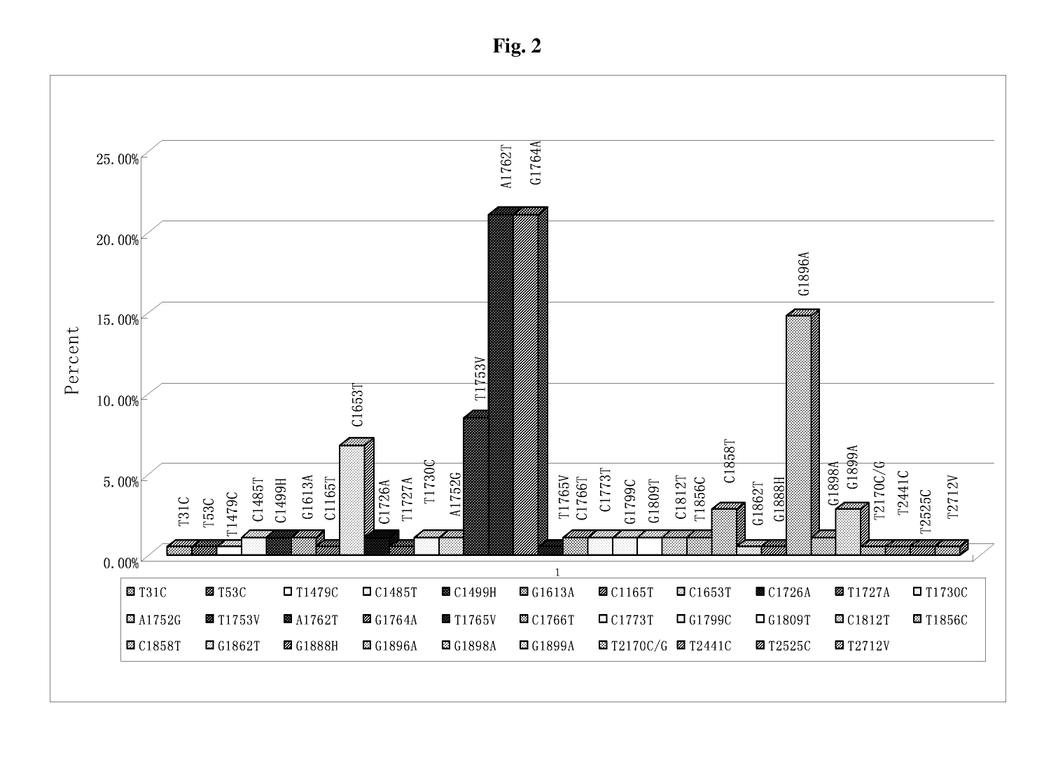

The inventors identified 32 distinct sites from total 176 repeatable count mutation sites selected from the 39 abovementioned literatures, and then these 32 mutation sites were analyzed statistically, based on frequency of occurrences for each mutation sites out of the total number of 176 mutation sits. The mutation sites from references statistic was present in FIG. 2. As a result, seven mutations was found with higher mutation frequency, which are C1653T, T1753V, A1762T, G1764A, C1858T, G1896A, G1899A, A1762T, G1764A and G1896A were with the highest mutation frequency among them. It was shown same mutation frequency (2.28%) for C1858T and G1899A. the mutation sites C1653T and T1753C showed higher mutation frequency, which were 7.34% and 8.47%, respectively.

The occurrence frequency of others 25 single mutation sites are between 0.56% and 1.13%. The total percent of the seven mutation sites covered 77.8% out of total mutation sites, as shown in Table 3.

TABLE 3

| Mutation Sites | C1653T | T1753C | A1762T | G1764A | C1858T | G1896A | G1899A |
|---|---|---|---|---|---|---|---|
| Frequency | 12 | 15 | 37 | 37 | 5 | 26 | 5 |
| Percentage (%) | 6.82% | 8.52% | 21.02% | 21.02% | 2.84% | 14.77% | 2.84% |

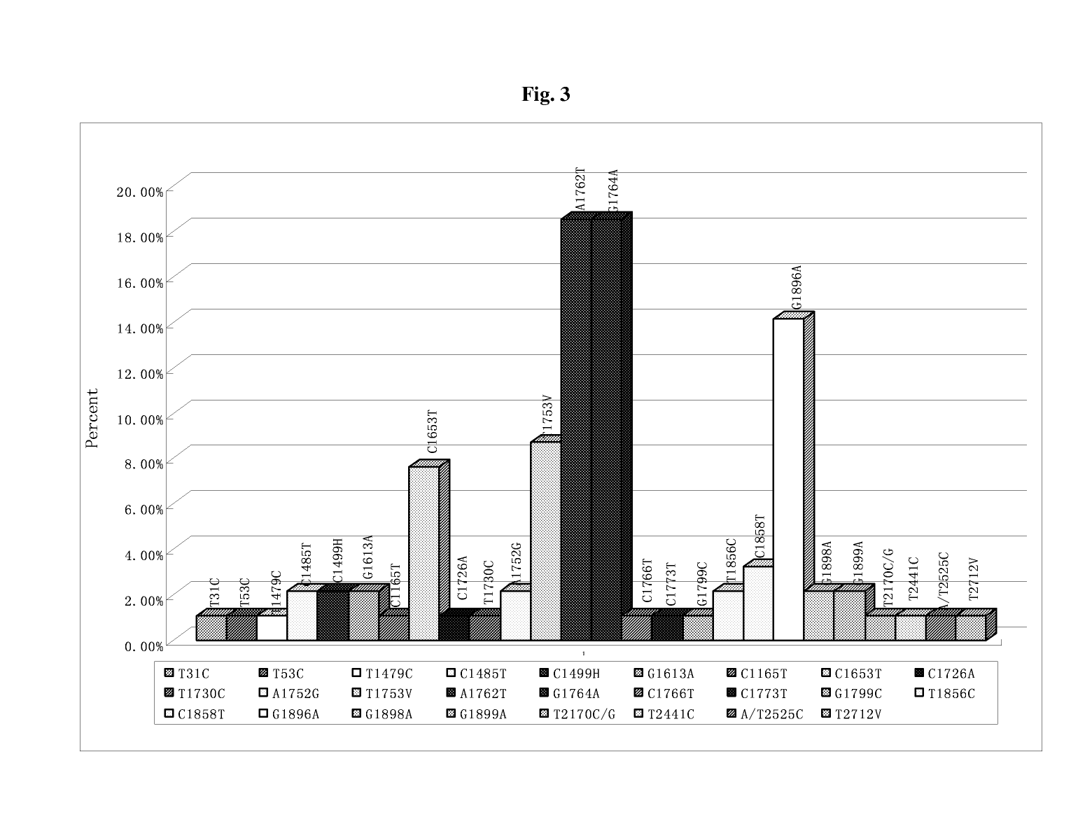

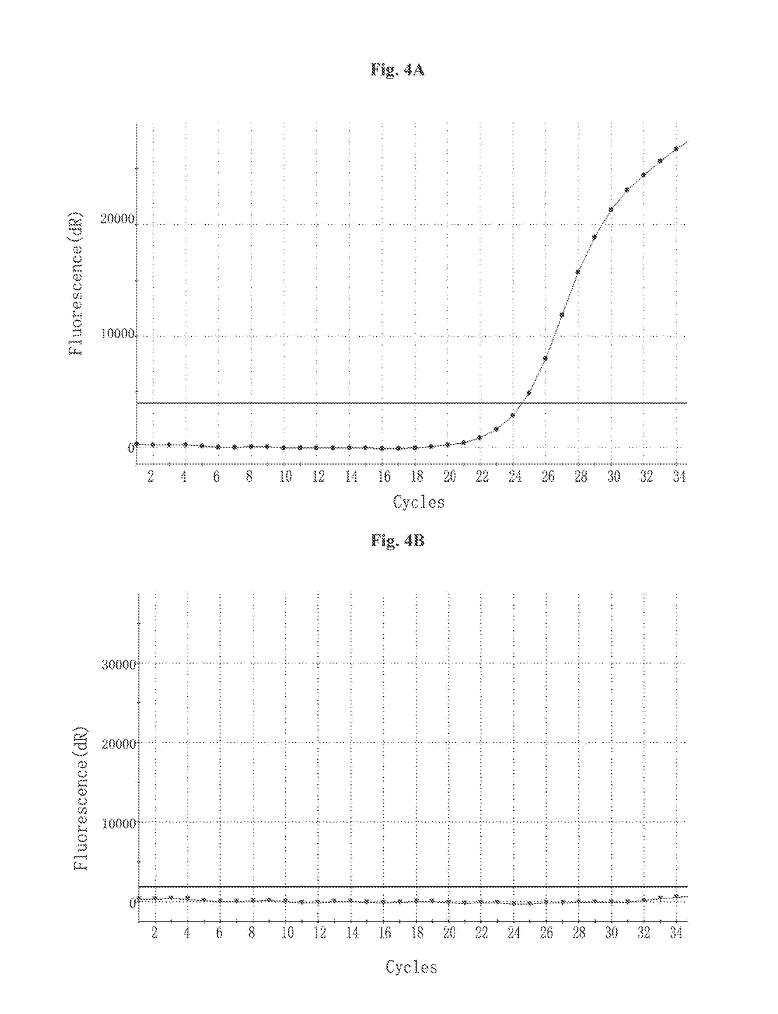

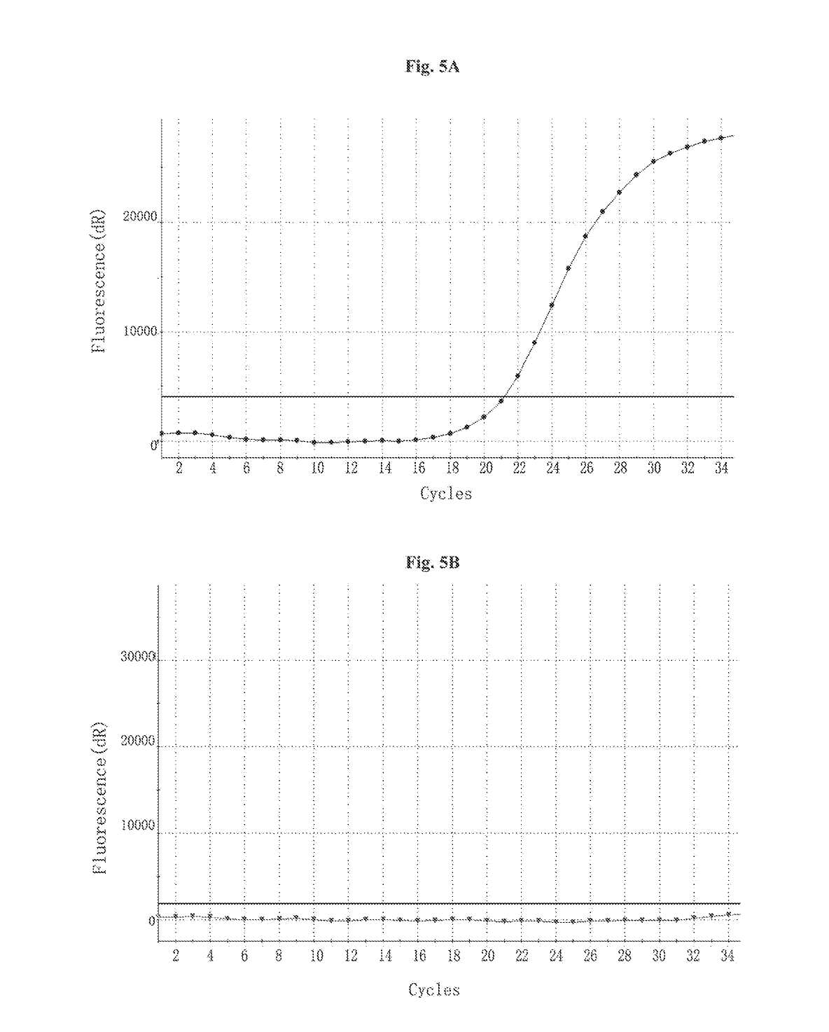

It was reported that over 70% belonged to C genotype and over 20% was B genotype in HCC patients in China study. (Meng et al., Chin Clin Oncol; 2007, 12, 435-440). Thus, the mutation rates of B/C genotypes of HBV were investigated separately. The inventors identified 26 different mutation sites from total 92 repeatable count mutation sits, and then these 26 mutation sites were analyzed statistically. Mutation sites C1653T, T1753C, A1762T, G1764A and G1896A showed the highest frequency, and total frequency covered 67% out of all mutations. Mutation rates of G1613A, T1856C and G1899A were 2.17% respectively. Other mutation rates were lower than 1.2%. The statistical results were present in FIG. 3.

At last, 11 mutation sites were identified for prediction risk of HCC by selection based on mutation frequency in HBV B/C genotypes and HBV all genotypes, which were C1653T, G1613A, T1753C, A1762T, G1764A, C1858T, G1896A, G1899A, C1485T, C1499A and T53C. The Chi-square tests at conventional level of 0.05 were performed to examine significance of these 11 mutations in HBV B/C genotypes and HBV all genotypes. The SPSS 13.0 software was used in the statistical tests, and the statistical results indicated that these 11 mutations were no significance differences in HBV B/C genotypes and HBV all genotypes. The results were shown in Table 5.

TABLE 5

| | 11 | Other | |
|---|---|---|---|
| | mutations | mutations | |
| HBV | frequency | frequency | P value |
| HBV all | 143 | 32 | 0.710 |
| genotypes | | | |
| HBV | 73 | 19 | |
| B/C genotypes | | | |

It was termed as wild-type nucleotides when a nucleotide appeared with the highest frequency in HBV after the alignment from the asymptomatic hepatitis B surface antigen carriers. It was termed as mutations that a nucleotide was substituted with three other nucleotides and deletion at each site.

In order to further investigate the frequency of abovementioned mutation sites associated with risk of HCC, an example experiment was performed by inventors.

### Example

In this example, eight mutations which are C1653T, A1762T, G1764A, T1753C, T1674C, C3116T, T53C and A1846T were analyzed for negative or positive association with prediction of HCC from HBV DNA sequences.

### Patients

Serum samples were collected from 85 patients with HBV-related hepatocellular carcinoma (HCC) and age-matched 85 HBV-infected patients who had not been diagnosed with HCC as control group.

### Extraction of DNA

Serum viral HBV DNA was extracted from 200 μl of serum using the AmoyDx Diagnostic Kit (AmoyDx, Xiamen, China) according to the manufacturer's instructions. Real-time PCR Amplification.

The primers and probes used in real-time fluorescence PCR were designed according to the eight mutation sites (C1653T, A1762T, G1764A, T1753C, T1674C, C3116T, T53C and A1846T) in HBV gene, and PCR detecting method was performed to detect the mutations. The specific primers for single mutation were designed, and only the corresponding mutation could be amplified. The PCR products could bind to probes, which issued a detectable fluorescent signal to identify the mutation type precisely. The primers and probes sequences were presented in Table 1. The compositions of real-time fluorescence PCR amplification as following:

| | | |
|---|---|---|
| DNA | 5 | μL |
| Primers | 1-5 | pmol |
| Probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |

Real-time PCR was carried out with a 3 min initial denaturation at 95° C., followed by 10 cycles of amplification (95° C. for 20 s, 65° C. for 20 s, and 72° C. for 20 s), then 35 cycles of amplification (95° C. for 20 s, 60° C. for 35 s, and 72° C. for 20s). The final 35 cycles are detected the FAM signal in anneal steps. Fluorescence PCR detection was performed using an MX3000P real-time PCR (SRATAGENE).

All the serum samples were conducted with direct DNA sequencing for detecting the eight mutations to confirm real-time PCR detected results. The results indicated our PCR detection shown high agreement with direct sequencing, the results was present in Table 6.

The experimental results indicated that the eight mutations showed significantly higher mutation rates in HCC patients than in control patients (p<0.0001), as shown in Table 6. The mutation rates of A1762T and G1764A were 74%, whereas they were 27% in the control group. The mutation rates of C3116T, C1653T and A1846T were 54%, 49% and 59%, respectively in HCC patients, whereas they were 11%, 8% and 14% in the control group. The mutation T1674C, T1753C and T53C were 41%, 38% and 42%, whereas they were 8%, 14% and 11% in the control group. The total odd ratio of eight mutations in HCC patients was 5.48 compared with control patients. The statistical significance was examined at the conventional level of 0.05 by Chi-square test. The statistical tests were performed by using the SPSS 13.0 software. The results of test were shown in Table 6.

The statistical results and experiment support the claim that the 14 mutations (A1858T, G1896A, G1899A, C1485T, C1499A, G1613A, T1753C, T1674C, C1653T, A1762T, G1764A, C3116T, T53C, and A1846T) are associated with susceptibility to development of HCC, Specifically, mutations C1653T, A1762T, G1764A, T1753C, T1674C, C3116T, T53C and A1846T have been identified as major candidate determinants of the susceptibility to HCC in human.

It is understood that the examples described herein are to illustrate the purpose only and that any changes and modifications in light thereof will be suggested to persons skilled in the art and are to be included within the spirit and purview of this application and scope of the appended claims.

TABLE 1

| | | | |
|---|---|---|---|
| Name | Nucleotide sequence (5′-3′) | Nt positions | Direction |

Primers and probes for C1653T

| | | | |
|---|---|---|---|
| C1653T-F | AGGAACCTGCCCAAGGTCTTGT | SEQ ID | sense |
| | | NO: 1 | |
| C1653T-R | CACCAACTCCTCCCACTCAGTA | SEQ ID | antisense |
| | | NO: 2 | |
| C1653T-P | FAM- | SEQ ID | Sense |
| | TCTTGGACTTTCAGCAATGTCAAC- | NO: 3 | |
| | BHQ1 | | |

Primers and probes for A1762T

| | | | |
|---|---|---|---|
| A1762T-F | CGGAGGAGGTTAGGTTAAT | SEQ ID | sense |
| | | NO: 4 | |
| A1762T-R | GGACATGAACATGAGATGATTAGGC | SEQ ID | antisense |
| | | NO: 5 | |
| A1762T-P | FAM- | SEQ ID | antisense |
| | GTGAAAAAGTTGCATGGTGCTGGT- | NO: 6 | |
| | BHQ1 | | |

Primers and probes for G1764A

| | | | |
|---|---|---|---|
| G1764A-F | CGGAGGAGGTTAGGTTAAAGA | SEQ ID | sense |
| | | NO:7 | |
| G1764A-R | GGACATGAACATGAGATGATTAGGC | SEQ ID | antisense |
| | | NO: 8 | |
| G1764A-P | FAM- | SEQ ID | antisense |
| | GTGAAAAAGTTGCATGGTGCTGGT- | NO: 9 | |
| | BHQ1 | | |

Primers and probes for T1753C

| | | | |
|---|---|---|---|
| T1753C-F | ACCTGCCCAAGGTCTTGCATAAGAG | SEQ ID | sense |
| | | NO: 10 | |
| T1753C-R | CCTCCTAGTACAAAGACCTTTAAC | SEQ ID | Antisense |
| | CTAG | NO: 11 | |
| T1753C-P | FAM- | SEQ ID | Sense |
| | GCAATGTCAACGACCGACCTTGAG- | NO: 12 | |
| | BHQ1 | | |

Primers and probes for A1846T

| | | | |
|---|---|---|---|
| A1846T-F | CCTCTGCCTAATCATCTCT | SEQ ID | sense |
| | | NO: 13 | |
| A1846T-R | CACAGAAGCTCCAAAT | SEQ ID | antisense |
| | | NO: 14 | |
| A1846T-P | FAM- | SEQ ID | sense |
| | ACTGTTCAAGCCTCCAAGCTG- | NO: 15 | |
| | BHQ1 | | |

Primers and probes for T1674C

| | | | |
|---|---|---|---|
| T1674C-F | AAGAGGACTCTTGGACTTC | SEQ ID | sense |
| | | NO: 16 | |
| T1674C-R | TCCACCAACTCCTCCCACTC | SEQ ID | antisense |
| | | NO: 17 | |
| T1674C-P | FAM- | SEQ ID | sense |
| | CAGCAATGTCAACGACCGACCT- | NO: 18 | |
| | BHQ1 | | |

Primers and probes for C3116T

| | | | |
|---|---|---|---|
| C3116T-F | TACTCACAACTGTGCCAGT | SEQ ID | sense |
| | | NO: 19 | |
| C3116T-R | AGGTGGAGATAAGGGAGTA | SEQ ID | antisense |
| | | NO: 20 | |
| C3116T-P | FAM- | SEQ ID | sense |
| | GCCTCCACCAATCGGCAGTCA- | NO: 21 | |
| | BHQ1 | | |

Primers and probes for T53C

| | | | |
|---|---|---|---|
| T53C-F | AGGAAGGCAGCCTACTCC | SEQ ID | sense |
| | | NO: 22 | |
| T53C-R | TGGAGCCACCAGCAGGAAG | SEQ ID | antisense |
| | | NO: 23 | |
| T53C-P | TCATCCTCAGGCCATGCAGT | SEQ ID | antisense |
| | | NO: 24 | |

TABLE 6

| | HCC | | Control | | | | Con- |
|---|---|---|---|---|---|---|---|
| | Muta- | | Muta- | | | | sis- |
| Position | tion | Wild | tion | Wild | p value | OR | tency |
| T1753C | 32 | 53 | 12 | 73 | P < 0.0001 | 5.48 | 94% |
| T1674C | 35 | 50 | 7 | 78 | P < 0.0001 | | 98% |
| C1653T | 42 | 43 | 7 | 78 | P < 0.0001 | | 95% |
| C3116T | 46 | 39 | 10 | 75 | P < 0.0001 | | 100% |
| A1846T | 50 | 35 | 23 | 62 | P < 0.0001 | | 100% |
| T53C | 36 | 49 | 18 | 67 | P < 0.0001 | | 98% |
| A1762T/ | 63 | 22 | 23 | 62 | P < 0.0001 | | 100% |
| G1764A | | | | | | | |

OR: Total odd ratio
Consistency: Consistency compared with direct sequencing

TABLE 1

| Study | Country | Mutation Sites | HBV genotype |
|---|---|---|---|
| Kim et al., 2008 | Korea | C1653T T1753V A1762T G1764A | C |
| Sakamoto et al., 2006 | Japan, | C1653T T1858HV A1762T G1764A C1862T | A, B, C |
| | Philippines | G1888H G1809T C1812T G1896A | |
| Yuan et al., 2007 | China | C1653T T1753V A1762T G1764A T1856C | B, C |
| | | G1898A C1858T G1896A G1899A | |
| Zhang et al., 2006 | China | A1762T G1764A | NA |
| Zhou et al., 2007 | China | A1762T G1764A G1896A | NA |
| Baptista et al., 1999 | South Africa | T1753V A1762T G1764A G1809T C1812T | NA |
| Blackberg et | Sweden, others | T1753V A1762T G1764A G1896A | A, B, C, D |
| al., 2003 | | | |
| Chen et al., 2006 | Taiwan | A1762T G1764A G1896A | B, C |
| Deng et al., 2004 | China | A1762T G1764A | NA |
| Ding et al., 2006 | China | A1762T G1764A | B, C |
| fang et al., 2002 | China | A1762T G1764A G1896A | B, C |
| Ito et al., 2006 | Japan, | C1653T T1753V A1762T G1764A G1896A | NA |
| | United States | | |
| Laskus et al., 1998 | Gambia | A1762T G1764A G1896A G1899A | NA |
| Liu et al., 2006 | Taiwan | A1762T G1764A G1896A | B, C |
| Livingston et | United States | A1762T G1764A G1896A | A, C, D, F |
| al., 2007 | | | |
| Muroyama et | Japan | C1485T A1762T G1764A G1896A | C |
| al., 2006 | | | |
| Mendy et al., 2008 | Gambia | A1762T G1764A G1896A | NA |
| Ni et al., 2003 | Taiwan | G1896A | NA |
| Shinkai et al., 2007 | Japan | C1653T T1753V A1762T G1764A C1485T | C2 |
| | | T1479C C1499H G1896A G1613A | |
| Song et al., 2005 | Vietnam | A1762T G1764A G1766A C1773T C1858T | NA |
| Sung et al., 2008 | Hong Kong | G1613A C1165T A1762T G1764A T31C | B, C |
| | | T53C | |
| | | C1499H T2170C/G G1899A T2441C | |
| | | A/T2525C T2712V | |
| Tanaka et al., 2006 | Japan, | C1653T T1753V A1762T G1764A T1765V | NA |
| | Hong Kong | G1896A G1899A | |
| Tanakashi et | Japan | C1653T T1753V A1762T G1764A | A, B, C, D |
| al., 1999 | | | |
| Tong et al., 2007 | United States | A1762T G1764A G1896A | A, B, C, D |
| Truong et al., 2007 | Japan, Vietnam | C1653T T1753V A1762T G1764A C1858T | C |
| | | G1896A | |
| Wang et al., 2007 | China | C1653T T1753V A1762T G1764A T1856C | B, C |
| | | G1898A C1858T G1896A G1899A | |
| Yuen et al., 2008 | Hong Kong | C1653T T1753V A1762T G1764A G1896A | B, C |
| Zhang et al., 2007 | China | A1762T G1764A | B, C, D |
| Chou et al., 2008 | China | C1726A T1753V A1762T G1764A G1799C | A, B, C |
| | | T1727A T1730C G1896A | |
| Guo et al., 2008 | China | C1653T T1753V A1762T G1764A C1766T | B, C |
| Kao et al., 2003 | Taiwan | A1752T T1753V A1762T G1764A C1773T | B, C |
| | | G1799C G1896A | |
| Zhang et al., 2007 | China | A1762T G1764A | NA |
| Zhu et al., 2008 | China | A1752G C1726A A1762T G1764A T1730C | C |
| Tong et al., 2007 | United states | A1762T G1764A G1896A | A, B, C |
| Jang et al., 2007 | Korea | A1762T G1764A G1896A | C |
| Yang et al., 2008 | Taiwan | A1762T G1764A G1896A | B, C |
| Liu et al., 2009 | China, Others | C1653T T1753V A1762T G1764A | A, B, C, D, F |
| Pisit et al., 2010 | Thailand | A1762T G1764A G1899A | NA |
| Zhang et al., 2010 | China | G1896A | NA |

NA: genotype undetermined.

TABLE 2

| Study | Country | Mutation Sites | HBV genotype |
|---|---|---|---|
| Kim et al., 2008 | Korea | C1653T T1753V A1762T G1764A | C |
| Yuan et al., 2007 | China | C1653T T1753V A1762T G1764A T1856C | B, C |
| | | G1898A C1858T G1896A G1899A | |
| Chen et al., 2006 | Taiwan | A1762T G1764A G1896A | B, C |
| Ding et al., 2006 | China | A1762T G1764A | B, C |
| fang et al., 2002 | China | A1762T G1764A G1896A | B, C |
| Liu et al., 2006 | Taiwan | A1762T G1764A G1896A | B, C |
| Muroyama et | Japan | C1485T A1762T G1764A G1896A | C |
| al., 2006 | | | |
| Shinkai et al., 2007 | Japan | C1653T T1753V A1762T G1764A C1485T | C |
| | | T1479C C1499H G1896A G1613A | |
| Sung et al., 2008 | Hong Kong | G1613A C1165T A1762T G1764A T31C T53C | B, C |
| | | C1499H T2170C/G G1899A T2441C A/T2525C | |
| | | T2712V | |
| Truong et al., 2007 | Japan, | C1653T T1753V A1762T G1764A C1858T | C |
| | Vietnam | G1896A | |
| Wang et al., 2007 | China | C1653T T1753V A1762T G1764A T1856C | B, C |
| | | G1898A C1858T G1896A G1899A | |
| Yuen et al., 2008 | Hong Kong | C1653T T1753V A1762T G1764A G1896A | B, C |
| Guo et al., 2008 | China | C1653T T1753V A1762T G1764A C1766T | B, C |
| Kao et al., 2003 | Taiwan | A1752T T1753V A1762T G1764A C1773T | B, C |
| | | G1799C G1896A | |
| Zhu et al., 2008 | China | A1752G C1726A A1762T G1764A T1730C | C |
| Jang et al., 2007 | Korea | A1762T G1764A G1896A | C |
| Yang et al., 2008 | Taiwan | A1762T G1764A G1896A | B, C |

What is claimed is:

1. A method of determining predisposition to cause hepatocelluar carcinoma for the patients infected with hepatitis B virus, wherein 14 nucleotide mutation sites of HBV genome are determined, comprising A1858T, G1896A, G1899A, C1485T, C1499A, G1613A, T1753C, T1674C, C1653T, A1762T, G1764A, C3116T, T53C and A1846T.

2. Biomarkers in hepatitis B virus genome of determining predisposition to cause hepatocelluar carcinoma for the patients infected with hepatitis B virus, the biomarks comprising A1858T, G1896A, G1899A, C1485T, C1499A, G1613A, T1753C, T1674C, C1653T, A1762T, G1764A, C3116T, T53C and A1846T mutation sites.

3. A method of determining eight mutations, comprising C1653T, A1762T, G1764A, T1674C, T1753C, C3116T, T53C and A1846T mutation sites of HBV genome associated with hepatocelluar carcinoma, comprising:
a) The primers and probes nucleotide sequence consist of SEQ ID NO: 1-SEQ ID: NO 24.
b) Detecting composition of real-time fluorescence PCR amplification as following:

| | | |
|---|---|---|
| DNA | 5 | μL |
| Primers | 1-5 | pmol |
| probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |

4. A method for amplication of eight mutations according claim 2, wherein the amplication comprising the follong steps: 1) initial denaturation at 95° C.; 2) first PCR amplication conditions are 10 cycles, each cycle comprising 95° C. for 20 s, 65° C. for 20 s, and 72° C. for 20 s; 3) second PCR amplication conditions are 35 cycles, each cycle comprising 95° C. for 20 s, 60° C. for 35 s, and 72° C. for 20s.

5. A method as claimed in claim 3, wherein the oligonucleotide primers and probes consist of the SEQ ID NO: 1-SEQ ID NO: 24. or an primers and probes oligonucleotide at least about 80-90% identical thereto.

6. A method as claimed in claim 3, wherein the oligonucleotide primers and probes consist of the SEQ ID NO: 1-SEQ ID NO: 24 or an oligonucleotide primers and probes at least about 70% identical sequence thereto.

7. The biomarkers as claimed in claim 1, wherein said 14 mutation sites comprising at least any one of them.

8. The biomarkers as claimed in claim claim 2, wherein said 14 mutation sites comprising at least any one of them.

9. A detecting kit as claimed in claim 2, wherein comprising the primers and probes nucleotide sequence consist of SEQ ID NO: 1-SEQ ID NO: 24 and composition including:

| | | |
|---|---|---|
| DNA | 5 | μL |
| Primers | 1-5 | pmol |
| probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |

10. A detecting kit as claimed in claim 3, wherein comprising the primers and probes nucleotide sequence consist of SEQ ID NO: 1-SEQ ID NO: 24 and composition including:

| | | |
|---|---|---|
| DNA | 5 | μL |
| Primers | 1-5 | pmol |
| probes | 1-5 | pmol |
| Taq DNA polymerase | 1-3 | U |
| Buffer | 5-20 | μL |
| dNTP | 100-300 | μM |
| MgCL₂ | 1-10 | mM |
| H₂O | 20-60 | μL |


SEQUENCE LISTING (24 sequences)

SEQ ID NO: 1 - DNA, 22 bp, Artificial Sequence
  Synthesized
aggaacctgcccaaggtcttgt

SEQ ID NO: 2 - DNA, 22 bp, Artificial Sequence
  Synthesized
caccaactcctcccactcagta

SEQ ID NO: 3 - DNA, 24 bp, Artificial Sequence
  Synthesized
tcttggactttcagcaatgtcaac

SEQ ID NO: 4 - DNA, 18 bp, Artificial Sequence
  Synthesized
cggaggaggtaggttaat

SEQ ID NO: 5 - DNA, 25 bp, Artificial Sequence
  Synthesized
ggacatgaacatgagatgattaggc

SEQ ID NO: 6 - DNA, 24 bp, Artificial Sequence
  Synthesized
gtgaaaaagttgcatggtgctggt

SEQ ID NO: 7 - DNA, 21 bp, Artificial Sequence
  Synthesized
cggaggaggttaggttaaaga

SEQ ID NO: 8 - DNA, 25 bp, Artificial Sequence
  Synthesized
ggacatgaacatgagatgattaggc

SEQ ID NO: 9 - DNA, 24 bp, Artificial Sequence
  Synthesized
gtgaaaaagttgcatggtgctggt

SEQ ID NO: 10 - DNA, 25 bp, Artificial Sequence
  Synthesized
acctgcccaaggtcttgcataagag

SEQ ID NO: 11 - DNA, 28 bp, Artificial Sequence
  Synthesized
cctcctagtacaaagacctttaacctag

SEQ ID NO: 12 - DNA, 24 bp, Artificial Sequence
  Synthesized
gcaatgtcaacgaccgaccttgag

SEQ ID NO: 13 - DNA, 19 bp, Artificial Sequence
  Synthesized
cctctgcctaatcatctct

SEQ ID NO: 14 - DNA, 16 bp, Artificial Sequence
  Synthesized
cacagaagctccaaat

SEQ ID NO: 15 - DNA, 21 bp, Artificial Sequence
  Synthesized
actgttcaagcctccaagctg

SEQ ID NO: 16 - DNA, 19 bp, Artificial Sequence
  Synthesized
aagaggactcttggacttc

SEQ ID NO: 17 - DNA, 20 bp, Artificial Sequence
  Synthesized
tccaccaactcctcccactc

SEQ ID NO: 18 - DNA, 22 bp, Artificial Sequence
  Synthesized
cagcaatgtcaacgaccgacct

SEQ ID NO: 19 - DNA, 19 bp, Artificial Sequence
  Synthesized
tactcacaactgtgccagt

SEQ ID NO: 20 - DNA, 19 bp, Artificial Sequence
  Synthesized
aggtggagataagggagta

SEQ ID NO: 21 - DNA, 21 bp, Artificial Sequence
  Synthesized
gcctccaccaatcggcagtca

SEQ ID NO: 22 - DNA, 20 bp, Artificial Sequence
  Synthesized
ctgccgttccggccgaccac

SEQ ID NO: 23 - DNA, 20 bp, Artificial Sequence
  Synthesized
tgagggtttaaatgtatacc

SEQ ID NO: 24 - DNA, 20 bp, Artificial Sequence
  Synthesized
tcatcctcaggccatgcagt